(19) United States
(12) Patent Application Publication
(10) Pub. No.: US 2022/0133599 A1
(43) Pub. Date: May 5, 2022

(54) METHODS AND COMPOSITIONS FOR ALTERING TEETH

(71) Applicant: The Texas A&M University System, College Station, TX (US)

(72) Inventors: Hong Liang, College Station, TX (US); Yan Chen, College Station, TX (US); Bradley Simon, College Station, TX (US); Lynne Opperman, College Station, TX (US)

(73) Assignee: The Texas A&M University System, College Station, TX (US)

(21) Appl. No.: 17/431,379

(22) PCT Filed: Feb. 17, 2020
(86) PCT No.: PCT/US2020/018523

§ 371 Date: Aug. 16, 2021

Related U.S. Application Data
(60) Provisional application No. 62/966,691, filed on Jan. 28, 2020.
(60) Provisional application No. 62/806,488, filed on Feb. 15, 2019.

(51) Int. Cl. A61K 6/20; A61K 6/70; A61K 6/60; A61C 5/20; A61C 19/06; A61D 5/00

(52) U.S. Cl. CPC A61K 6/20; A61K 6/70; A61D 5/00; A61C 5/20; A61C 19/066; A61K 6/60

(57) ABSTRACT

Embodiments of the present disclosure pertain to methods of altering a tooth by applying an object to the tooth, where the object includes a composition that is released from the object onto the tooth, and where the composition has an altering effect on the tooth, such as tooth whitening, tooth repair, tooth maintenance, or combinations thereof. Additional embodiments of the present disclosure pertain to the objects of the present disclosure. The objects of the present disclosure may be in a chewable form, such as in the form of a chew toy or a chewing gum. The objects of the present disclosure may also be in a form that is capable of being applied topically by rubbing or brushing. The objects of the present disclosure may also include a second composition that has effects other than tooth altering effects, such as enticing a chewing of the object.

### CROSS-REFERENCE TO RELATED APPLICATIONS

This application claims priority to U.S. Provisional Patent Application No. 62/806,488, filed on Feb. 15, 2019, and U.S. Provisional Patent Application No. 62/966,691, filed on Jan. 28, 2020. The entirety of each of the aforementioned applications is incorporated herein by reference.

### BACKGROUND

Traditional methods of dental repair have numerous limitations. For instance, many dental repair procedures for animals involve costly, stressful and dangerous surgical operations. Additional dental repair procedures require bio-mineralization and re-mineralization, which require a long time to take effect. Methods of addressing the discoloration of teeth also have numerous limitations, such as potential harm to the tooth enamel. As such, alternative materials and methods are needed to restore and whiten teeth. Numerous embodiments of the present disclosure address the aforementioned needs.

### SUMMARY

In some embodiments, the present disclosure pertains to methods of altering a tooth. In some embodiments, the methods of the present disclosure occur by applying an object to the tooth, where the object includes a composition that is released from the object onto the tooth. The composition has an altering effect on the tooth, such as tooth whitening, tooth repair, tooth maintenance, or combinations thereof. Additional embodiments of the present disclosure pertain to the objects of the present disclosure.

In some embodiments, the objects of the present disclosure are in a chewable form, such as in the form of a chew toy or a chewing gum. In some embodiments, the objects of the present disclosure are in a form that is capable of being applied topically by rubbing or brushing.

In some embodiments, the compositions of the present disclosure include, without limitation, compositions with tooth whitening effects, compositions with tooth repair effects, compositions with tooth maintenance effects, or combinations thereof. In some embodiments, the compositions of the present disclosure include amine-intercalated zirconium phosphate. In some embodiments, the compositions of the present disclosure are in the form of particles, such as amine-intercalated zirconium phosphate particles.

In some embodiments, the objects of the present disclosure also include a second composition that has effects other than tooth altering effects. For instance, in some embodiments, the second composition is capable of enticing chewing of the object. In some embodiments, the second composition includes, without limitation, a flavoring agent, a coloring agent, a texturing agent, mixers, or combinations thereof.

The objects of the present disclosure may be applied to a tooth in various manners. For instance, in some embodiments, the application process includes, without limitation, chewing the object, rubbing the object onto the tooth, brushing the object onto the tooth, rinsing the tooth with the object, or combinations thereof. In some embodiments, the application process includes chewing the object in such a manner that the composition is released onto the tooth as the object is chewed. In some embodiments, the application process includes rubbing or brushing the object onto the tooth in such a manner that the composition is released onto the tooth as the object is rubbed or brushed onto the tooth.

In some embodiments, the composition forms a solid film on a surface of the tooth. In some embodiments, the film has a thickness ranging from 100 nm to 5 μm. In some embodiments, the film is in the form of a tribofilm on a surface of the tooth.

The methods and objects of the present disclosure can have various altering effects on a tooth. For instance, in some embodiments, the altering effect includes tooth maintenance, such as protection from cracks, improvement of surface integrity, prevention of tooth breakdown, providing antimicrobial properties, or combinations thereof. In some embodiments, the altering effect includes tooth repair, such as the filling of cracks, tooth restoration, or combinations thereof. In some embodiments, the altering effect includes tooth whitening.

The methods of the present disclosure may be utilized to apply the objects of the present disclosure to the teeth of various subjects. For instance, in some embodiments, the subjects include, without limitation, dogs, cats, rats, gerbils, hamsters, guinea pigs, rabbits, humans, or combinations thereof. In some embodiments, the subjects include dogs. In some embodiments, the subjects include humans.

### DESCRIPTION OF THE DRAWINGS

FIG. 1A illustrates a method of altering a tooth.

FIG. 1B depicts an object for altering a tooth.

FIG. 2 provides an image that summarizes a tribo-mastication process. Two canine teeth were used in this set up: the “disc” tooth and the “pin” tooth. The “disc” tooth was sealed in epoxy (dashed block) with the surface enamel polished. The “pin” tooth was pressed down and rubbed against the “disc” tooth with a reciprocal motion. The grey materials represent the location of the repairing agent.

FIG. 3 shows the x-ray mass attenuation coefficient for Zr. The data used in this plot was collected from the National Institute of Standards and Technology database (NIST).

FIG. 4 shows an interferometer image of the enamel wear track generated from the tribo-mastication process with simulated food (bone mill). The scale bar size was 100 μm.

FIG. 5 shows interferometer images of an enamel surface after a tribo-mastication process with a repairing agent, including S0 (FIG. 5A), S2.5 (FIG. 5B), S5 (FIG. 5C) and S10 (FIG. 5D).

FIG. 6 shows atomic force microscopy (AFM) height (FIGS. 6A and 6C) and phase (FIGS. 6B and 6D) images of the tribofilm generated with S0 (FIGS. 6A and 6B) and S5 (FIGS. 6C and 6D). The unit of the color bars was nm in FIGS. 6A and 6C, and mV in FIGS. 6B and 6D.

FIG. 7 shows the Raman spectra collected from the enamel surface and the tribofilm generated from S0 and S5. Two peaks resulted from the phosphate group.

FIG. 8 shows a three-dimensional rendering of a pin tooth after a tribo-mastication process with repairing agent S5. The distribution of the Zr element calculated from the dual energy k-edge technique was rendered as the golden color. FIG. 8A shows the tip of the tooth. FIG. 8B shows the cross-sectional rendering showing the repairing agent entered the surface crack. Scale bar length is 4 μm.

FIG. 9 shows a scratch test result from the tribofilm generated by S5. The red colored part was the tribofilm. After the scratching, part of the film still exists while a deep groove was formed on the enamel substrate.

FIG. 10 illustrates a dogtooth repaired according to an aspect of the present disclosure.

FIG. 11 illustrates an image showing topography of a dog tooth after being repaired.

FIG. 12 illustrates repairing film generation on dog teeth.

FIG. 13 illustrates a profile of dog teeth.

FIG. 14 illustrates that scratching does not remove a film from a tooth.

FIG. 15 illustrates film generation on human teeth.

FIG. 16 illustrates an area being coated by a film.

FIG. 17 illustrates that scratching does not remove a film from a tooth.

FIG. 18 illustrates an image of a film built on a tooth.

FIG. 19 illustrates proposed materials that have better wear resistance and hardness (The chart was adapted from: Materials Selection in Mechanical Design, 3rd Edition, Michael F. Ashby).

FIG. 20 illustrates the whitening of a human tooth according to an aspect of the present disclosure.

FIG. 21 illustrates a comparison of whitened tooth and standard whitening scales. The tooth inside the glass tube was whitened by rubbing.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Tooth enamel is the hardest and most dense structure in the body. Tooth enamel enables grinding up of food before swallowing and protects the interior of the tooth from normal oral bacteria and other harmful oral substances.

Tooth damage is a major health threat that can deteriorate the quality of life. In extreme cases, tooth damage can be life threatening. Although teeth are the hardest tissue in the body, they can still suffer from mechanical attritions and erosions from acid. Untreated, those damages may cause the loss of teeth or infections.

In addition, when an enamel layer is removed, it can cause exposure of sensitive dentin to environmental factors. This can in turn result in dental pain and infections.

Causes for dentin exposure or disruption of the enamel layer include, but are not limited to, trauma, periapical inflammation, enamel hypoplasia, dysplasia, hyper-mineralization, heredity disorders (e.g., amylogenesis imperfecta or dentiongenesis imperfect), or dental disease. In fact, dental disease has been a prevalent disease reported in both dogs and cats.

In animals such as dogs, extensive chewing is closely related to teeth damage. In fact, the enamel layer is significantly thinner in dogs than in humans and is therefore more prone to excessive wear. Such wear can in turn lead to dentin and pulp exposure, thereby causing pain. For instance, United States pet-owners report that corrective dental procedures are extremely painful and require pain medications to be administered in approximately 92% of cases.

Traditional dental repair processes involve surgical operations to apply restorative materials. However, such operations can be costly and stressful. In addition, the restorative materials can cause the occlusal wear of the treated teeth.

Moreover, to perform dental restoration or repair in veterinary medicine for damaged enamel, general anesthesia is routinely required. However, the administration of general anesthesia can predispose animals to anesthetic related death and owners to emotional and financial distress.

An alternative to the currently used dental restoration processes has been re-mineralization of the damaged tooth enamel. In particular, bio-mineralization processes were used to apply hydroxyapatite nanoparticles to teeth and form hard enamel tissue in vitro or in vivo. Additional re-mineralization precursors have included casein phosphopeptide-stabilized amorphous calcium phosphate, amelogenin with fluoride, and polydopamine with hydroxyapatite (HAP). Unlike traditional dental treatments, the aforementioned methods can repair teeth with materials that were almost identical to the teeth itself.

However, bio-mineralization and re-mineralization processes require a long time to take effect. This presents a major challenge to the clinical application of such prolonged processes because they usually do not match with the rate of tooth wear. Moreover, many of the materials utilized for bio-mineralization and re-mineralization processes are not resistant to high bite forces, especially when chewing on hard materials.

Another related tooth related issue has been the discoloration of teeth. There are many materials and methods used by dentists and individuals that have been utilized for whitening teeth. However, many of the aforementioned materials and methods are harmful to enamel and health of teeth. Adverse effects associated with these materials and methods include tooth sensitivity, gingival or mucosal irritation, oxidation of enamel or dentine, disruption of restorative materials, a reduction in shear and tensile bond strength, local inflammation, and tooth sensitivity.

As such, alternative materials and methods are required to restore and whiten teeth. Numerous embodiments of the present disclosure address the aforementioned needs.

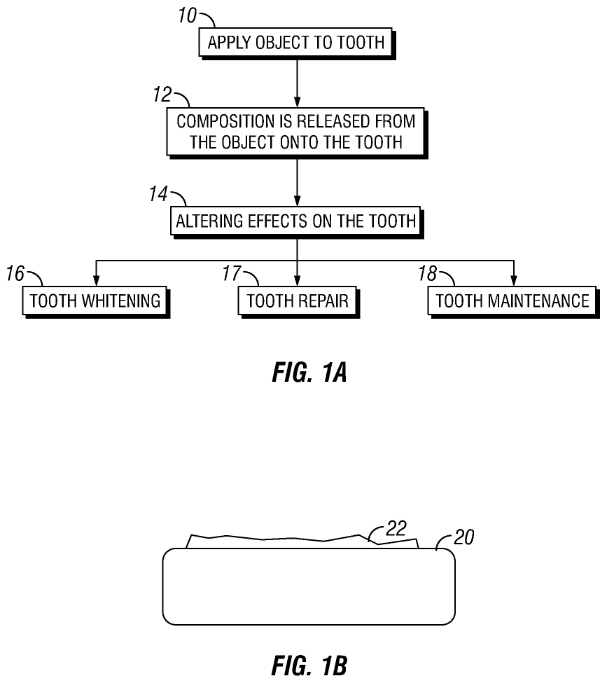

In some embodiments, the present disclosure pertains to methods of altering a tooth. In some embodiments illustrated in FIG. 1A, the methods of the present disclosure include applying an object that includes a tooth altering composition onto the tooth (step 10). Thereafter, the composition is released from the object onto the tooth (step 12). The composition can have various altering effects on the tooth, including tooth whitening (step 16), tooth repair (step 17), and tooth maintenance (step 18).

Additional embodiments of the present disclosure pertain to objects that include tooth altering compositions. In some embodiments illustrated in FIG. 1B, the objects of the present disclosure include object 20, which includes tooth altering composition 22 that is releasable from object 20 onto a tooth. Composition 22 can have various altering effects on a tooth, including tooth whitening, tooth repair, and tooth maintenance.

As set forth in more detail herein, the methods and objects of the present disclosure can have numerous embodiments. In particular, various objects and various compositions may be applied to various teeth in various manners in order to have various altering effects on the teeth.

Objects

In the present disclosure, objects generally refer to materials that can carry the compositions of the present disclosure and release them onto a tooth. The objects of the present disclosure can be in various forms. For instance, in some embodiments, the objects of the present disclosure include, without limitation, porous materials, woods, fabrics, chewable objects, chew toys, gums, objects capable of being applied topically by rubbing or brushing, pastes, liquids, rinses, or combinations thereof.

In some embodiments, the objects of the present disclosure are in a chewable form. For instance, in some embodiments, the objects of the present disclosure are in the form of a chew toy. In more specific embodiments, the objects of the present disclosure are in the form of chew toys that can help dogs, cats, and other mammals alter teeth through chewing. In some embodiments, the chew toys resemble standard chew todays, except that they include the compositions of the present disclosure. In some embodiments, the chew toys are coated with the compositions of the present disclosure or made from the compositions of the present disclosure. In some embodiments, the chew toy are three-dimensional (3D)-printable chewing toys.

In some embodiments, the objects of the present disclosure are in the form of a gum. In more specific embodiments, the objects of the present disclosure are in the form of chewing gums that can help humans alter teeth through chewing. In some embodiments, the chewing gums resemble standard chewing gums, except that they include the compositions of the present disclosure. In some embodiments, the chewing gums are coated with the compositions of the present disclosure or made from the compositions of the present disclosure.

In some embodiments, the objects of the present disclosure are in a form that is capable of being applied topically by rubbing or brushing. For instance, in some embodiments, the objects of the present disclosure are in the form of a tooth paste that can help humans or other animals alter teeth through applying the tooth paste onto teeth. In some embodiments, the tooth paste resemble standard tooth paste, except that they include the compositions of the present disclosure. In some embodiments, the tooth paste are coated with the compositions of the present disclosure or made from the compositions of the present disclosure.

Compositions

The objects of the present disclosure may include various compositions. For instance, in some embodiments, suitable compositions can include compositions that can have altering effects on a tooth. In some embodiments, the compositions include, without limitation, compositions with tooth whitening effects, compositions with tooth repair effects, compositions with tooth maintenance effects, or combinations thereof.

In some embodiments, the compositions of the present disclosure include, without limitation, hydrogen peroxide, carbamide peroxide, fluoride containing materials, phosphorous containing materials, calcium phosphate, zirconium phosphate (ZrP), α-zirconium phosphate, γ-zirconium phosphate, titanium phosphate, γ-titanium phosphate, iron oxide, zirconium oxide, zirconium dioxide, hydroxyapatite (HAP), kaolinite, bentonite, gold, silver, silica, ceria, alumina, zirconia, calcium aluminate, boron carbide, silicon carbide, silicon nitride, iron oxide, magnesium oxides, zinc chloride, sodium fluoride, hydrated salts thereof, amine-intercalated materials thereof, or combinations thereof.

In some embodiments, the compositions of the present disclosure include an amine-intercalated zirconium phosphate. In some embodiments, the amine is an amine-based polymer.

In some embodiments, the amine-based polymer includes, without limitation, polyetheramines, Jeffamines, Jeffamine M600, or combinations thereof.

In some embodiments, the compositions of the present disclosure include an amine-intercalated zirconium phosphate and zirconium dioxide. In some embodiments, the compositions of the present disclosure include an amine-intercalated zirconium phosphate, zirconium dioxide, and calcium phosphate. In some embodiments, the compositions of the present disclosure include zirconium dioxide at concentrations of up to 40 wt % of the composition.

The compositions of the present disclosure can have various organic and inorganic materials at various mass ratios. For instance, in some embodiments, the compositions of the present disclosure have an organic:inorganic mass ratio of about 0.1:1 to about 0.1:100. In some embodiments, the compositions of the present disclosure have an organic:inorganic mass ratio of about 0.1:1 to about 1:2. In some embodiments, the compositions of the present disclosure have an organic:inorganic mass ratio of about 1:2.

In some embodiments, the compositions of the present disclosure have an organic:inorganic mass ratio of about 0.1:100 to about 1:1. In some embodiments, the compositions of the present disclosure have an organic:inorganic mass ratio of about 0.1:100. In some embodiments, the compositions of the present disclosure have an organic:inorganic mass ratio of about 1:1. In some embodiments, the compositions of the present disclosure have an organic:inorganic mass ratio of about 4:1.

The compositions of the present disclosure may be in various forms. For instance, in some embodiments, the compositions of the present disclosure may be in the form of particles, sheets, layered structures, or combinations thereof.

In some embodiments, the compositions of the present disclosure are in the form of particles. In some embodiments, the particles have diameters that range from about 100 nm to about 5 μm. In some embodiments, the particles have diameters that range from about 100 nm to about 1 μm. In some embodiments, the particles have diameters of about 1 μm.

In some embodiments, the compositions of the present disclosure are in the form of particles that include, without limitation, calcium phosphate particles, zirconium dioxide particles, hydroxyapatite particles, kaolinite particles, bentonite particles, cloisite particles, gold particles, silver particles, silica particles, ceria particles, alumina particles, zirconia particles, calcium aluminate particles, boron carbide particles, silicon carbide particles, silicon nitride particles, iron oxide particles, magnesium oxide particles, zinc chloride particles, sodium fluoride particles, zirconium phosphates particles, zirconium phosphate particles (e.g., amine intercalated zirconium phosphate particles), titanium phosphate particles, hydrated salts thereof, or combinations thereof. In some embodiments, the compositions of the present disclosure are in the form of zirconium phosphate particles.

In some embodiments, the compositions of the present disclosure are in the form of hydroxyapatite and Jeffamine M-600-intercalated zirconium phosphate particles. In some embodiments, the mass ratio of hydroxyapatite and Jeffamine M-600-intercalated zirconium phosphate particles range from about 0:1 to about 1:1. In some embodiments, the mass ratio of hydroxyapatite and Jeffamine M-600-intercalated zirconium phosphate particles are 0:1, 0.25:1, 0.5:1, or 1:1.

In some embodiments, the compositions of the present disclosure are in the form of hydroxyapatite and amine intercalated zirconium phosphate particles. In some embodiments, the compositions of present disclosure are in the form of titanium phosphate particles. In some embodiments, the compositions of the present disclosure are in the form of zirconium dioxide and amine intercalated zirconium phosphate particles.

In some embodiments, the compositions of the present disclosure are in encapsulated form. In some embodiments, the encapsulated forms include, without limitation, dissolvable nanospheres, microspheres, capsules, or combinations thereof.

In some embodiments, the compositions of the present disclosure are in the form of a layered structure. In some embodiments, the layered structure contains from about 2 layers to about 20 layers. In some embodiments, the layered structure contains from about 2 layers to about 10 layers. In some embodiments, the layers in the layered structure are at least held together by Van der Waals forces.

In some embodiments, the compositions of the present disclosure are functionalized with a functionalizing agent. In some embodiments, the functionalizing agent is an organic molecule. In some embodiments, the organic molecule includes, without limitation, polymers, amine-based polymers, polyethylene glycols, surfactants, erythritol, sorbitol, glycerol, flavorants, triclosan, sodium lauryl sulfate, or combinations thereof. In some embodiments, the organic molecules include, without limitation, Jeffamines (ED-600, ED-400), polyethylene glycol (PEG) copolymers (e.g., mPEG₁₂-NH₂, mPEG₆-NH₂, surfactants (e.g., sulfate, sulfonate, phosphate, carboyxlates, docusate, PFOS, perfluorobutanesulfonate, PFOA, PFO, CTAB, CPC, BAC, BZT, DODAB, and the like), erythritol, sorbitol, flavorants, triclosan, sodium lauryl sulfate, glycerol, or combinations thereof. In some embodiments, the organic molecules include a surfactant.

The compositions of the present disclosure may be associated with the objects of the present disclosure in various manners. For instance, in some embodiments, the compositions of the present disclosure are coated on a surface of the objects of the present disclosure. In some embodiments, the compositions of the present disclosure are intertwined with the objects of the present disclosure. In some embodiments, the compositions of the present disclosure are dispersed within the objects of the present disclosure.

In some embodiments, the compositions of the present disclosure may be encapsulated within the objects of the present disclosure. In some embodiments, the compositions of the present disclosure may be encapsulated within the objects of the present disclosure in the form of dissolvable nanospheres, microspheres, capsules or other materials. In more specific embodiments, the compositions of the present disclosure are encapsulated within a chewable toy or gum (e.g., a soft center inside some gums), or encapsulated within dissolvable nanospheres, microspheres, capsules or other materials within the object (e.g., chewables, pastes or liquids).

Second Compositions

In some embodiments, the objects of the present disclosure may also include second compositions. Second compositions generally refer to compositions that have effects other than tooth altering effects. For instance, in some embodiments, the second compositions of the present disclosure are capable of enticing chewing of an object (e.g., chewable object). In some embodiments, the second compositions of the present disclosure include, without limitation, a flavoring agent, a coloring agent, a texturing agent, mixers, or combinations thereof.

In some embodiments, the second compositions of the present disclosure include mixers. In some embodiments, the mixers can include, without limitation, hydroxyapatite, calcium fluoride, zirconia, silica, calcium aluminate, zinc oxide eugenol, zinc oxide, polycarboxylate, sodium alginate, polyether, silicones, agar, calcium hydroxide, glass ionomer, zirconium dioxide, titanium dioxide, barium sulfate, ytterbium, amalgam, composite resin, dental compomer, dimethacrylate monomer, difunctional resin, porcelain, acrylic, or combinations thereof.

Application of Objects to Tooth

The compositions of the present disclosure may be applied to tooth in various manners. For instance, in some embodiments, the compositions of the present disclosure are applied to tooth by chewing the objects, rubbing the objects onto the tooth, brushing the objects onto the tooth, rinsing the tooth with the objects, or combinations thereof.

In some embodiments, the applying of the objects onto a tooth occurs by chewing the objects. In some embodiments, the composition is released onto the tooth as the object is chewed.

In some embodiments, the applying of the objects onto a tooth occurs by rubbing or brushing the object onto the tooth. In some embodiments, the composition is released onto the tooth as the object is rubbed or brushed onto the tooth.

The objects of the present disclosure can be applied to various regions of a tooth. For instance, in some embodiments, the objects of the present disclosure are applied on a surface of a tooth. In some embodiments, the objects of the present disclosure are applied onto a tooth enamel.

The application of the objects of the present disclosure to a tooth can have various structural effects on a tooth. For instance, in some embodiments, the composition forms a solid film on a surface of the tooth as a result of the application. In some embodiments, the film forms a thickness on a surface of the tooth. In some embodiments, the film has a thickness ranging from 100 nm to 5 μm. In some embodiments, the film has a thickness of up to about 1 μm. In some embodiments, the film is in the form of a uniform film. In some embodiments, the film is in the form of a tribofilm. In some embodiments, the formed tribofilm is in the form of a continuous smooth film. In some embodiments, the formed tribofilm consists of organic and inorganic materials.

The compositions of the present disclosure may be applied onto a tooth through various mechanisms. For instance, in some embodiments, the compositions of the present disclosure are applied onto a tooth through a tribo-mastication process. In some embodiments, the composition is exfoliated and broken down by tribo-mechanical force.

In some embodiments, the application of the objects of the present disclosure onto a tooth results in the polymerization of the compositions of the present disclosure on the tooth. For instance, in some embodiments, any polymer chains attached to the compositions of the present disclosure become polymerized during the application process. In some embodiments, the application of the objects of the present disclosure onto a tooth results in the aggregation (e.g., particles aggregation) of the compositions of the present disclosure through Van der Waals forces.

Tooth Altering Effects

The compositions of the present disclosure can have various tooth altering effects. For instance, in some embodiments, the compositions of the present disclosure provide tooth whitening, tooth repair, tooth maintenance, or combinations thereof.

In some embodiments, the compositions of the present disclosure provide tooth maintenance. In some embodiments, the compositions of the present disclosure provide tooth maintenance by protecting tooth from cracks, improving the surface integrity of tooth, preventing tooth breakdown, providing antimicrobial properties, or combinations thereof.

In some embodiments, the compositions of the present disclosure provide tooth repair. In some embodiments, the tooth repair include the filling of any cracks on the tooth, tooth restoration, or combinations thereof.

In some embodiments, the compositions of the present disclosure provide tooth whitening. In some embodiments, tooth whitening includes the elimination of any stains on the tooth, the appearance of a white color on the tooth, or combinations thereof.

In some embodiments, the compositions of the present disclosure provide the tooth altering effects without the requirement of any follow-up procedures, such as shaping, grinding, or polishing.

Subjects

In some embodiments, the objects of the present disclosure are applied to the tooth of a subject. The objects of the present disclosure may be applied to the tooth of various subjects. For instance, in some embodiments, the subjects include, without limitation, dogs, cats, rats, gerbils, hamsters, guinea pigs, rabbits, humans, or combinations thereof. In some embodiments, the subject includes a dog. In some embodiments, the subject includes a human.

Applications and Advantages

In some embodiments, the methods and objects of the present disclosure provide a commercially available and convenient alternative to currently used dental restoration processes that require general anesthesia. For instance, in some embodiments, the compositions of the present disclosure can be applied to commercially available chew toys or other chewable objects. Accordingly, during normal chewing, the compositions of the present disclosure can rebuild the damaged teeth without presenting the risks, costs and distress associated with general anesthesia.

Moreover, in some embodiments, the methods and objects of the present disclosure can reduce the incidence of dental disease, dental infection, and dental pain through self-restoration, and reduce the risk and distress associated with general anesthesia. Moreover, in some embodiments, the methods and objects of the present disclosure can provide affordable, convenient, and effective alternatives to invasive techniques that are currently available for tooth repair and restoration.

For instance, in some embodiments, the objects and compositions of the present disclosure provide an enamel restoring material that does not require hospitalization, general anesthesia, or a dentist (e.g., a veterinary dentist). In more specific embodiments, pets can restore their enamel at home in a stress-free environment by chewing on an object.

Moreover, in some embodiments, the objects and methods of the present disclosure provide low-cost, long-lasting, and biocompatible alternatives to keeping teeth white and healthy. Moreover, the objects and methods of the present disclosure can be simple to use. For instance, in some embodiments, the objects and methods of the present disclosure can be used at home on a daily basis through chewing the objects, rinsing the objects in the mouth, or utilizing the objects as tooth paste.

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Nanomaterials for Dental Restoration

In this Example, Applicants demonstrate that various compositions are effective in repairing teeth. Applicants' prior research found that α-zirconium phosphate (α-ZrP) is an effective additive to form tribofilm (Dai et al., Lubricants 2016, 4 (3), 28; Xiao et al., Appl. Surf Sci. 2015, 329, 384-389; He et al., Colloids Surf Physicochem. Eng. Asp. 2014, 452, 32-38; and Chen et al., J. Tribol. 2018, 141 (3)). Particularly, Applicants found that α-zirconium phosphate nanoparticles had a unique layered structure. Moreover, there were Van der Waals forces between each layer that held the layers together. Under shear, such particles exfoliate, thereby resulting in low friction.

Chemical reactions between particle and surface were triggered by mechanical force. To date, teeth repairing or surface modification using the concept of tribofilms has not been reported. In this Example, Applicants report a novel approach to restore, modify, and protect tooth enamel using functionalized nanoparticles. This new method was inspired by the effectiveness of tribochemical interactions between rubbing surfaces. Applicants designed dental restoration materials through formation of tribofilms and nanoparticle-polymer biomineralization. Results show that a solid film of phosphorus mineral was successfully rubbed on worn teeth with thicknesses of up to 1 μm. Such films are also effective in filling tooth surface cracks and improving surface integrity.

### Example 1.1. Materials

All inorganic reagents used in this Example were purchased from Sigma Aldrich. The organic amine M-600 used in this research was provided by the Huntsman Corporation. The synthesis of the materials in this research used the following procedures. The α-ZrP nanoparticles and HAP nanoparticles were synthesized with hydrothermal methods. For α-ZrP, 50 ml food grade 12M phosphoric acid and 5 g Zirconyl chloride octahydrate (ZrOCl₂.8H₂O) were mixed under constant stirring conditions. The mixture was then sealed in a PTFE lined autoclave and moved to a 200° C. oven for 24 hours.

The precursor of HAP was prepared with the following procedure: 12 ml 0.25 M Calcium nitrate solution (Ca(NO₃)₂) was added dropwise into 20 ml 0.15 M disodium hydrogen phosphate (Na₂HPO₄) solution under constant stirring and 50-60° C. The pH of this mixture was then adjusted to 8-10 with ammonium hydroxide. This mixture was placed in a PTFE lined autoclave and placed in a 160° C. oven for 12 hours. After the hydrothermal reactions, the products were washed with DI water and retrieved by centrifugation three times.

The washed nanoparticles were dried in a vacuum furnace at 70° C. for 12 hours. The synthesized α-ZrP was intercalated with polyetheramine M-600. The synthesized 1 mmol α-ZrP was first dispersed in 5 ml of DI water with an ultrasonic bath for 1 hour. Next, 0.4 M of an M-600 solution was added into the dispersion dropwise.

The tribo-repair paste was a mixture between the HAP nanoparticles and M-600 amine intercalated α-ZrP. The synthesized dispersion of intercalated α-ZrP was first mixed with the HAP nanoparticles. The amount of HAP nanoparticles used in mass ratios to α-ZrP before intercalation was 0:1, 0.25:1 and 0.5:1 (the produced repairing agents were labeled as S0, S2.5 and S5, respectively). The product was centrifuged for 10 minutes. After the centrifuge process, the supernatant was removed by tilting the tube, and the precipitate was retrieved. Those precipitates were then used in the tribo-mastication process described in the following sections.

All dog canine teeth were obtained from the Texas A&M veterinary medical teaching hospital. Before the experiment, the teeth were washed in hydrogen peroxide (H₂O₂) and deionized (DI) water. The residual soft tissue was removed with a small knife.

A “disc” sample was made from the canine tooth for the experiment. To make the sample, the canine tooth was first sealed in the epoxy resin disc with the distal surface facing up. Then, the enamel of this surface was carefully exposed and grinded with sandpaper and polished with a 3 μm sized diamond paste.

A beef bone mill was used to simulate the mastication process during hard food eating. The beef split femur bone was cooked in a high pressure cooker for 4 hours and air dried. The cooked bone was then grinded with a mortar and pestle and then mixed with DI water in a 1:1 mass ratio.

### Example 1.2. Experimental Procedure

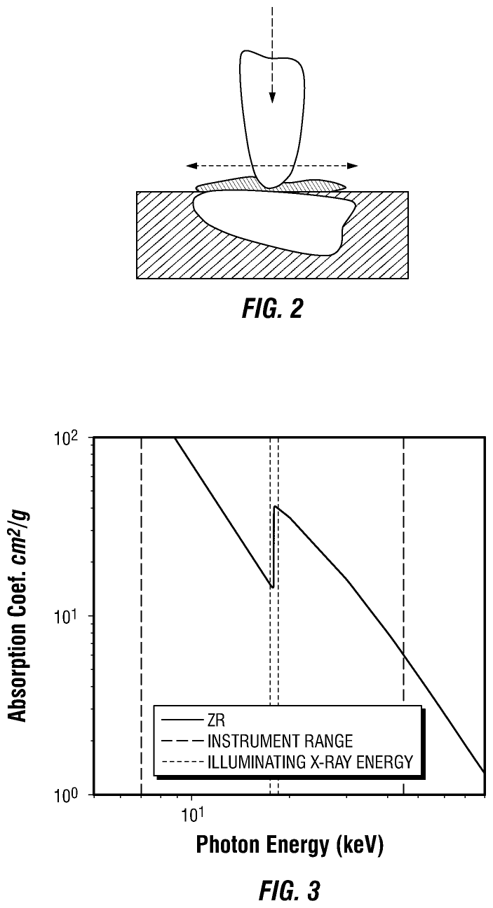

To simulate chewing and grinding motion of teeth, a tribo-mastication process was used. This process was conducted on a pin-on-disc tribometer (CSM instruments). The set-up of this tribo-mastication experiment is illustrated in FIG. 2. Two dog canine teeth were used in this set-up. One of the teeth was used as a pin, and another was used as a disc. The normal force on the pin was 1N. The pin tooth was moved with a sinusoid reciprocal motion on the disc tooth with an amplitude of 2 mm and a maximum speed of 1 cm/s.

The repair agent was placed between the tooth tribopair before the tribo-mastication process. The motion was terminated after 100 cycles. After the process, samples were washed with DI water and air dried.

A scratch test was then preformed on the sample to qualitatively evaluate the film's mechanical performance. The same tribometer with a steel needle was used for this scratch test. The needle was pressed against the tooth disc sample with 1N force and scratched across the formed tribofilm manually.

After the tribo-film deposition, the effect of the repairing agent was characterized. The formed tribofilm morphology and microstructure were characterized by an interferometer (Zygo NewView 600, Zygo Crop) and AFM (Nano-R2, Pacific Nanotechnology) close-contact mode. The Raman spectrum result of the tribofilm was collected with iHR550 Spectrometer (HORIBA Scientific, Edison, N.J.) with a 532 nm leaser. Two spectra were collected, one from the tribofilm, and other from the polished teeth surface.

The pin tooth that went through the tribo-mastication process was then analyzed with Synchrotron micro X-ray CT (μ-XCT). These experiments were performed on the Beam line 8.3.2 instrument at Lawrence Berkeley National Laboratory. All samples were imaged with a LuAG:Ce scintillator. Tomographic reconstruction was conducted using Xi-CAM with a tomopy tomography plugin.

The imaging of the coating was accomplished with a dual energy k-edge technique. Before the test, the illuminating x-ray energy was calibrated to the x-ray absorption edge of the Zr with a pure α-ZrP nanoparticle sample. As illustrated in FIG. 3, the x-ray absorption by Zr element jumps around 18 KeV. Two illuminating energies were used to take two separate tomographic images of the same sample: 18.2 KeV and 17.8 KeV.

As illustrated in FIG. 3, when the sample was illuminated with 17.8 KeV, the Zr element was brighter than the one illuminated with 18.2 KeV. Thus, a subtraction between two data set can reveal the distribution of the Zr element which was the distribution of Applicants' repairing tribofilm. The data collected from the experiments were then rendered with the Avizo software.

### Example 1.3. Results and Discussion

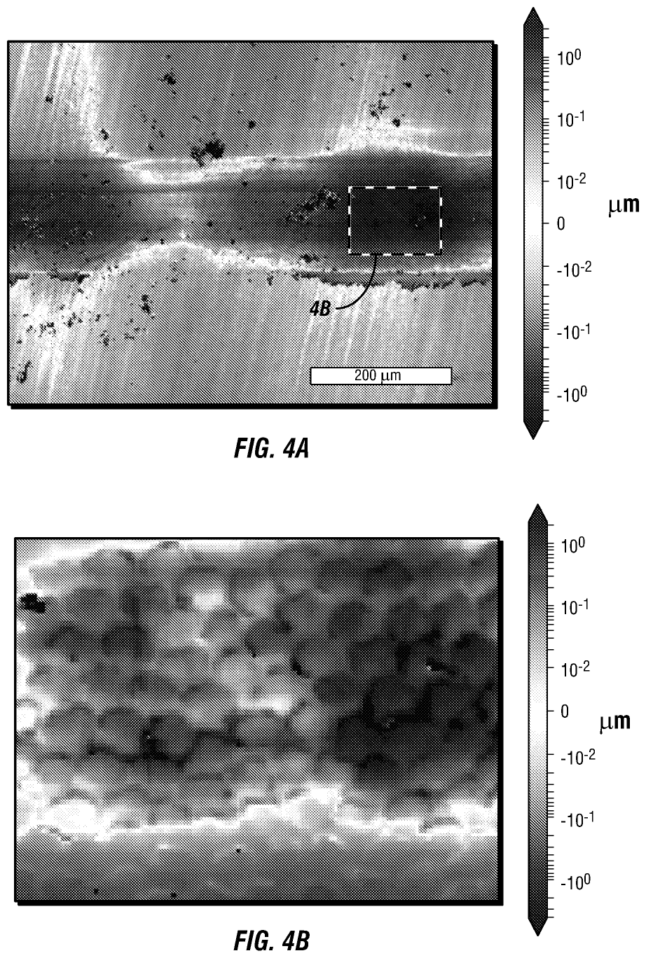

When the tooth sample went through the tribo-mastication process with the simulated food, the enamel wear occurred. The interferometer image of the wear track is shown in FIGS. 4A-B. Inside the wear track, the enamel rod was protruding out. It appears that the soft tissue was removed by the abrasion with bone mill particles. Despite the similar chemical composition (HAP), the bone mill cannot attach to the enamel surface with this process.

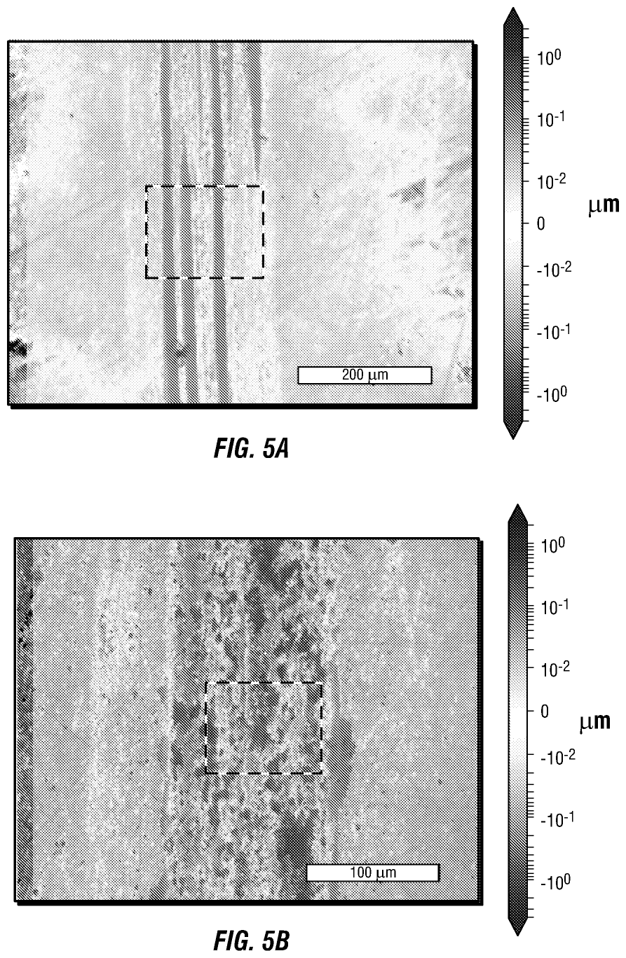

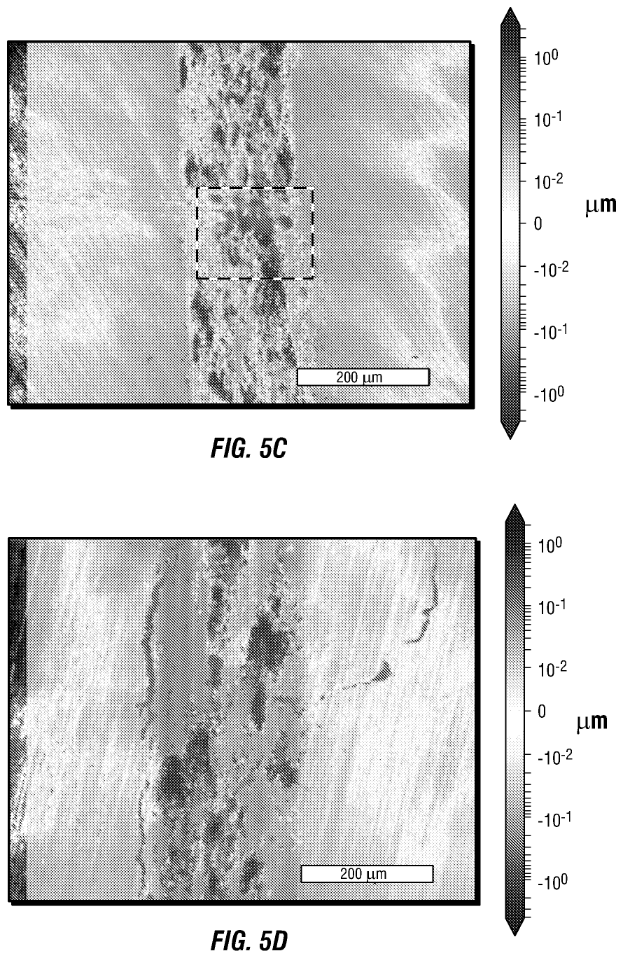

Tribofilms were formed after the tribo-mastication process for all repairing agents. The interferometer topographic image is shown in FIGS. 5A-D. The film shown in the center of FIGS. 5A-D was the result of the tribo-mastication process conducted with the repairing agent.

Without the involvement of additives, the tribological pin-on-disc wear will cause a groove generated on the surface of the disc. Here, a film with the thickness range from 100 nm to 1 micron was generated instead. The pressure that generates this film was calculated to be in the range of 100 MPa. The generation of this film prevented wear on the tooth.

The thickness and coverage of this tribofilm can be controlled with the amount of HAP nanoparticles. With the increased amount of HAP, the thickness of the generated tribofilm was increased. With repairing agent S0, the film thickness was only around 100 nm, but increased to 2 μm when the HAP:ZrP mass ratio was increased to 1:1 in the case of S10. The coverage of the tribofilm was also changed by this mass ratio.

In the case of S0, a continuous film was produced, the sample S2.5 and S5 can also generate tribofilms almost fully cover the wear track. Furthermore, when the ratio of ZrP and HAP increased to 1:2 in mass ratio, the tribofilm cannot be formed at all. The addition of too much HAP nanoparticles can also cause the repairing agent to lose its tribo-film forming property. Because of this, Applicants concluded that the S5 is a good compromise between the film coverage and film thickness.

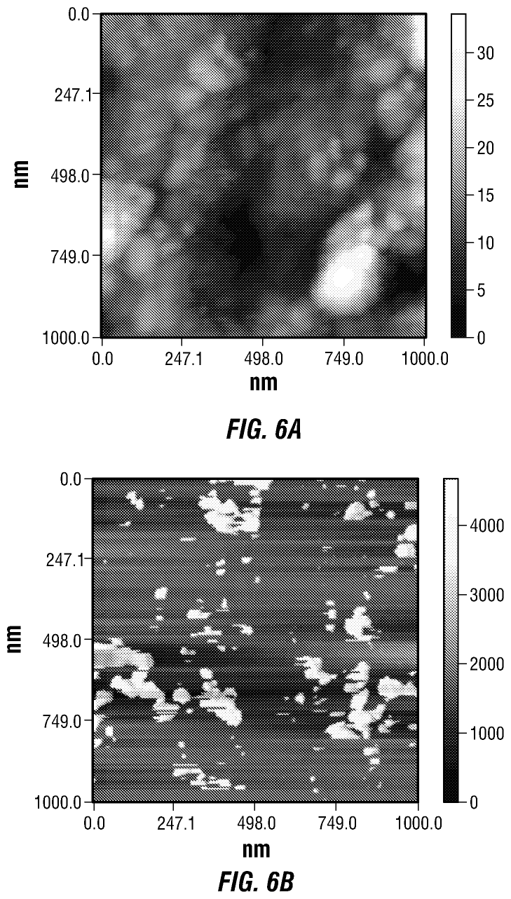

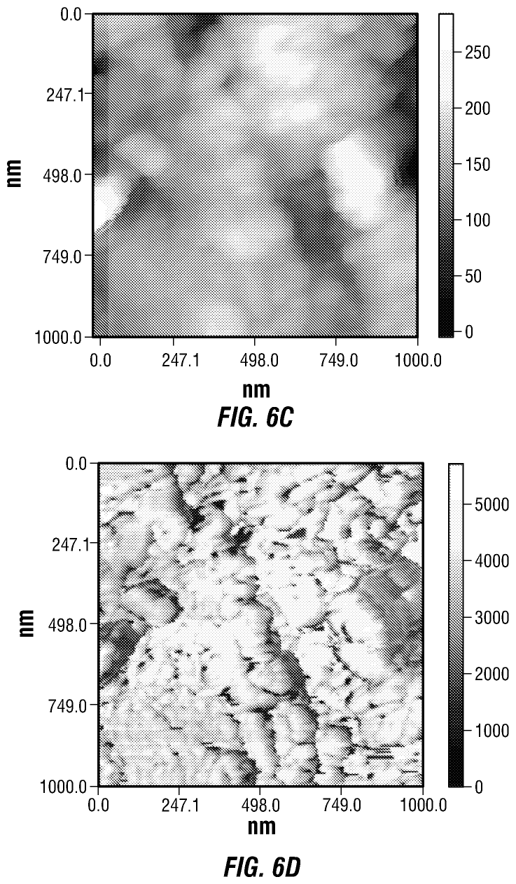

As shown in FIGS. 6A-D, an atomic force microscopy (AFM) image revealed the micro-structure of the intercalated α-ZrP tribofilm. The AFM height and phase map of the tribofilm in FIGS. 5A and 5C is shown. The tribofilm generated with S0 consisted of many flaky particles with a size around 200 nm (FIGS. 6A-B). The results indicate that the deposition was not a direct deposition of the ZrP particles, which has a size of around 1 μm. Because the intercalation process decreased the Van der Waals force between the ZrP layers, they can be more readily exfoliated from the shear force during the tribo-mastication process. The exfoliated and functionalized two-dimensional sheets thus became the building blocks of the tribofilm.

The inclusion of HAP nanoparticle changed the micro-structure of the resulting tribofilm. Instead of flaky aggregation of particles, a more regular cellular like structure was formed (FIGS. 6C-D). This cellular structure consisted of grains with almost identical sizes, very likely to be the HAP nanoparticles. The HAP nanoparticles survived the tribo-mastication process, and were adhered together by the M600 attached to ZrP.

The aforementioned results further explain why the inclusion of HAP nanoparticles increased the thickness of the film but decreased the coverage. In this Example, the HAP cannot form tribofilm without the intercalated α-ZrP. Thus, the higher concentration of HAP will decrease the possibility of intercalated α-ZrP contacting with the tooth surface.

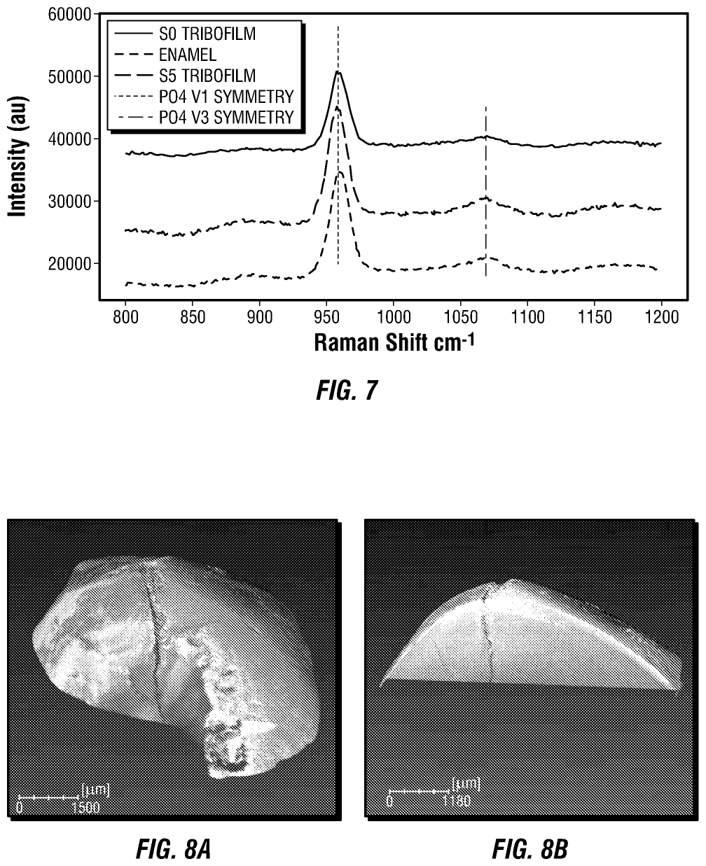

The generated film did not introduce any foreign inorganic functional groups on the surface of the teeth. The tribo-chemical process was known to chemically alter the phosphate group. The Raman result showed only two peaks for both tooth enamel and tribofilms (FIG. 7). Both peaks resulted from the —PO₄ group. No additional chemicals were detected from the Raman spectrum.

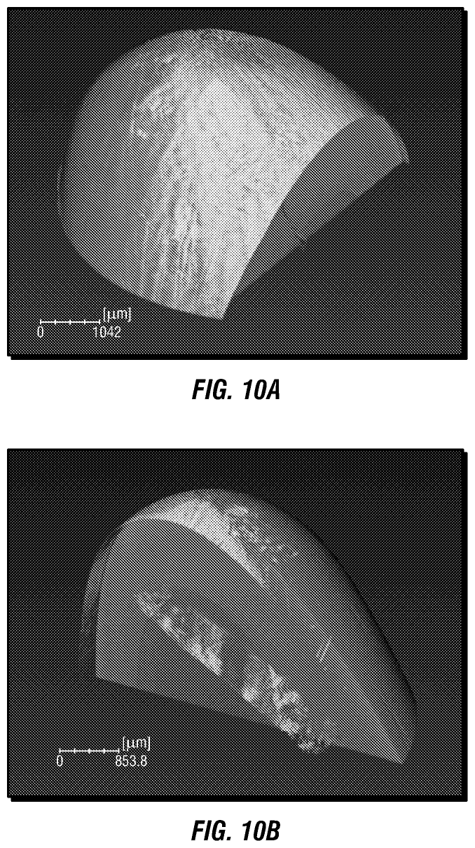

In order to characterize the repairing ability of the repairing agent on the whole teeth instead of only the surface, micro X-ray CT was used. As shown in FIGS. 10A-B, the density distribution of the Zr element was overlaid on the reconstructed tooth image. The tooth was the tip of the pin canine tooth after the tribo-mastication process with the S5 agent. A tooth with a crack on its tip was selected for this Example. In the contacting area, a film was formed that wrapped on the tip of the tooth. In addition, Applicants found the Zr element presented inside the cracks of the tip.

Based on the aforementioned results, Applicants propose a tribofilm forming mechanism for the tooth repairing agent in this Example for the pure intercalated α-ZrP. The nanoparticles were exfoliated and broken down by the tribo-mechanical force. Thereafter, the polymer chains attached to the exfoliated nanoparticles reattached those exfoliated layers under shear.

When polymer chains grind with the mechanical force, the polymerization process can occur. This mechanochemical polymerization process could be the driving force of the growth of tribofilm.

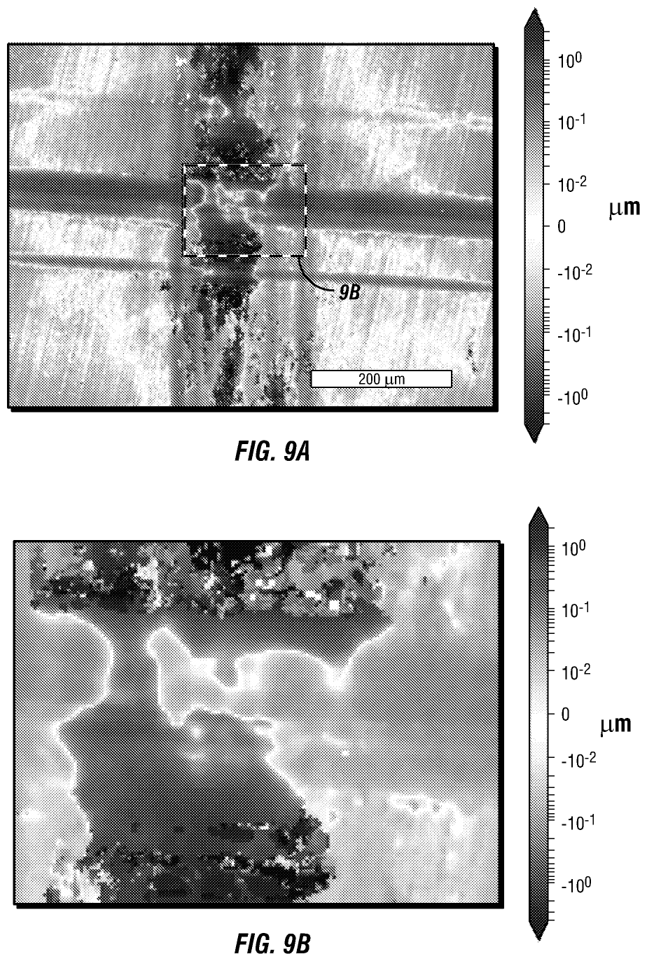

When HAP nanoparticle was introduced, the polymer chain further interacts with the HAP crystal, and adheres to the HAP crystals on the tooth surface, thereby forming a thick coherent tooth-repairing tribofilm. Under the scratch test, the generated films' scratch resistance was equal to or higher than the property of the tooth itself (FIGS. 9A-B). The scratching from the steel needle caused the loss of materials from both the tooth surface and the tribofilm surface. However, the tribofilm was not removed or exfoliated from the sample's surface despite the scratching. When compared to the deep groove on the unprotected enamel surface, this tribofilm shows good hardness and wear resistance.

### Example 1.4. Conclusions

In this Example, Applicants provide a simple one-step procedure to repair and maintain tooth of humans and pets. The repair and formation of a protective film were made possible through mechanical rubbing (chewing) of materials consisting of nanoparticles, polymers, and biomaterials for mineralization. After the simulated tribo-mastication between two teeth, the tooth repair agent can form a tribo-film with a thickness up to 2 μm. The formed repairing film has hardness comparable to the enamel surface. In addition to surface repairs, this new agent can enter the cracks on the enamel surface.

Furthermore, Applicants found that the polyether modified nanoparticles can induce the formation of tribofilms in aqueous solutions. The formation of such protective film may be attributed to the affinity between the polyether and HAP inside the tooth enamel.

### Example 2. Compositions for Tooth Self-Restoration

In this Example, Applicants provide a chewable object that includes a composition associated with the chewable object, where the composition is released onto a tooth as the chewable object is chewed. In this Example, the composition was an aqueous paste of HAP nanoparticles and M-600 amine intercalated ZrP nanoparticles. In this Example, Applicants also provide a method of applying a material to a tooth, where the method includes chewing a chewable object including a composition, where the composition is released onto the tooth as the chewable objected is chewed.

This Example describes designs and fabricate nanomaterials that can heal the worn spots on a tooth through chewing. The self-repairing or self-protecting function is based on the mechano-catalytic, tribo-chemical, or mechano-chemical properties of nanomaterials. The dental self-restoration can be accomplished by chewing-triggered chemical reactions between nanoparticles, enamel, bone, and calcium-containing compounds. During a chewing process, a solid, durable, and highly wear-resistant thick coating can be generated without any follow-up procedures such as shaping, grinding, or polishing.

As shown in FIGS. 10A-B, a dog tooth was repaired by rubbing the compositions of the present disclosure for a few minutes. The image was obtained through micro-tomography and the orange color was the trace of Zr in the film. The element went through cracks and grooves on the dog tooth and covered uniformly across.

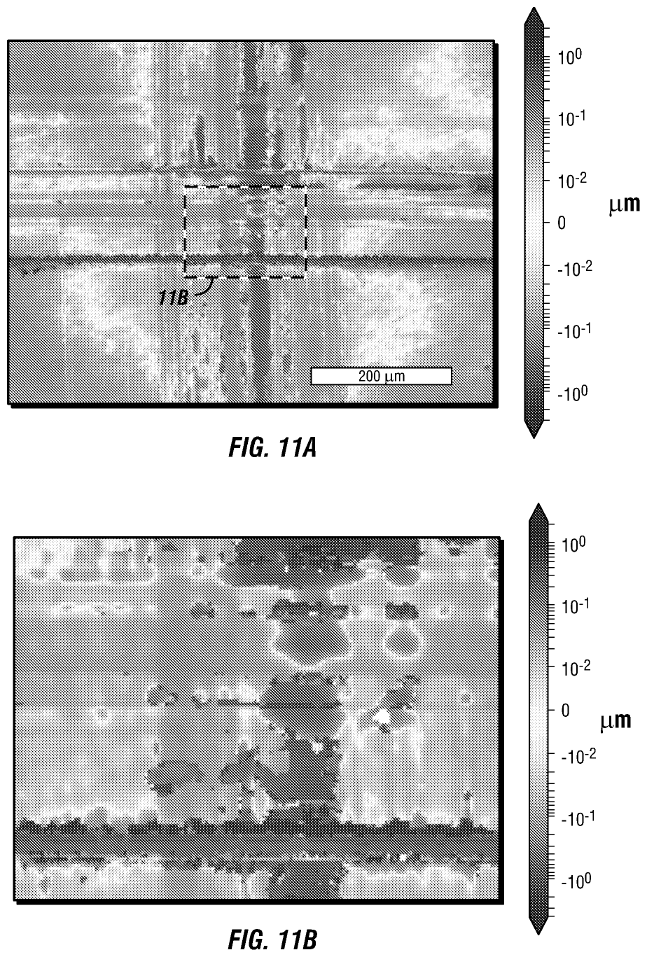

The repairing film is durable and scratch resistant. FIGS. 11A-B illustrate images showing topography of a dog tooth after being repaired. The rubbing direction is conducted via up-down application. The surface is built up due to rubbing. After that, a scratch test is conducted across the rubbing area. The scratch test shows that the naked tooth is scratched while the film remains to be in place.

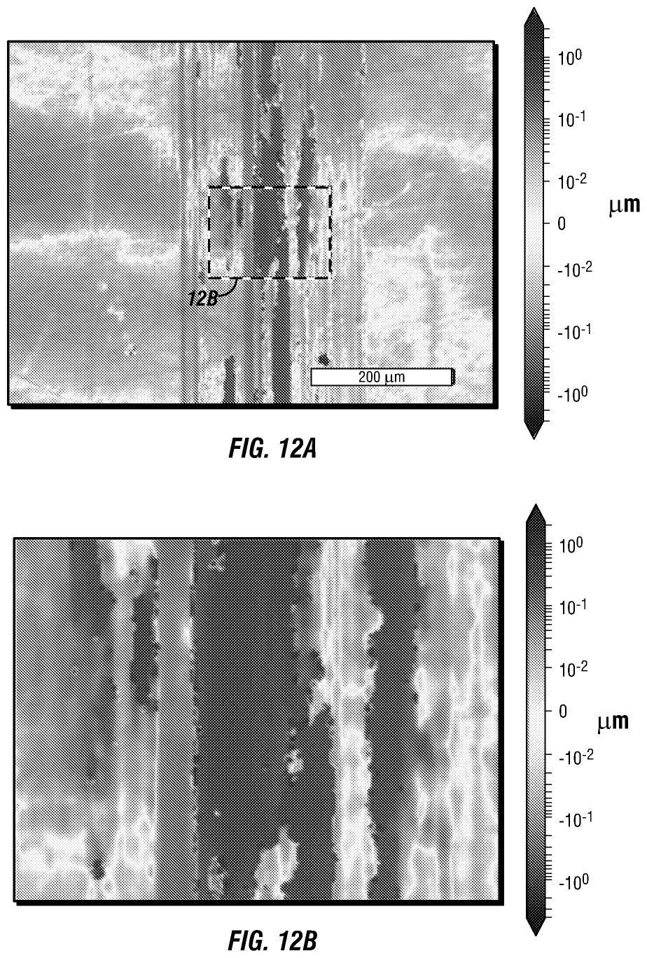

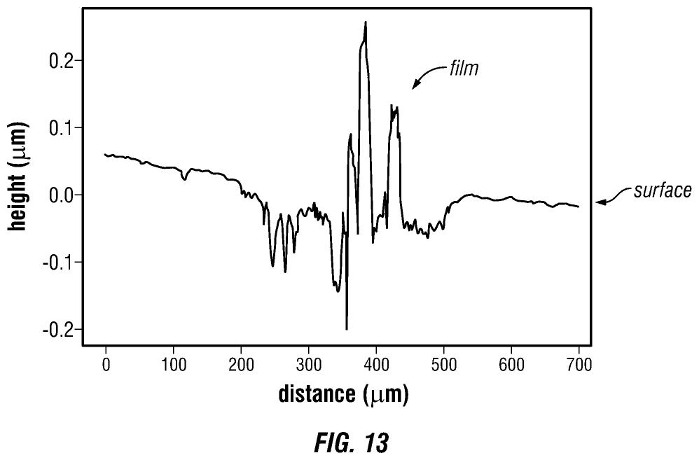

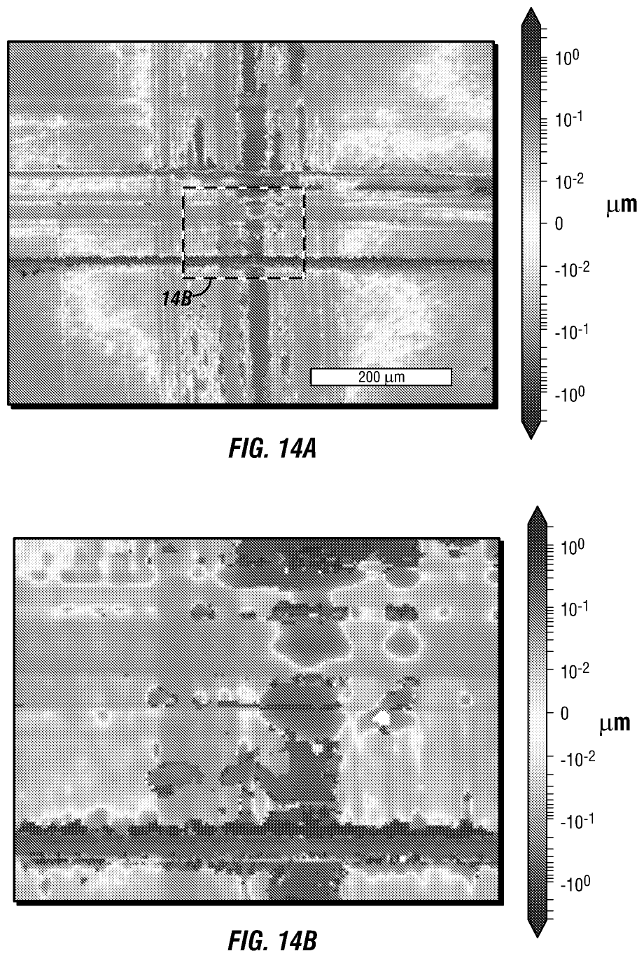

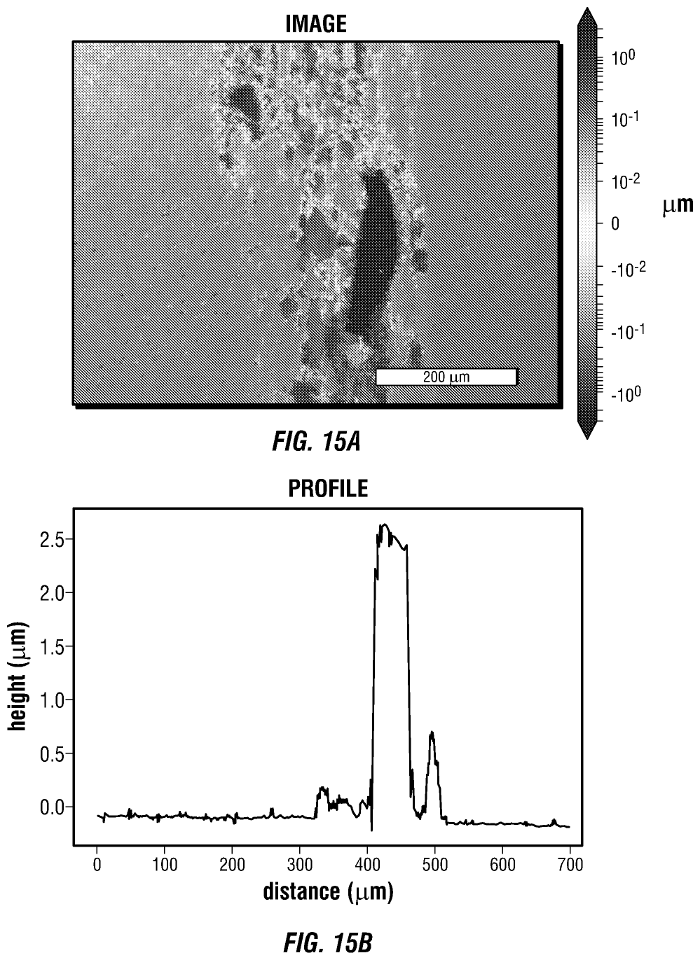

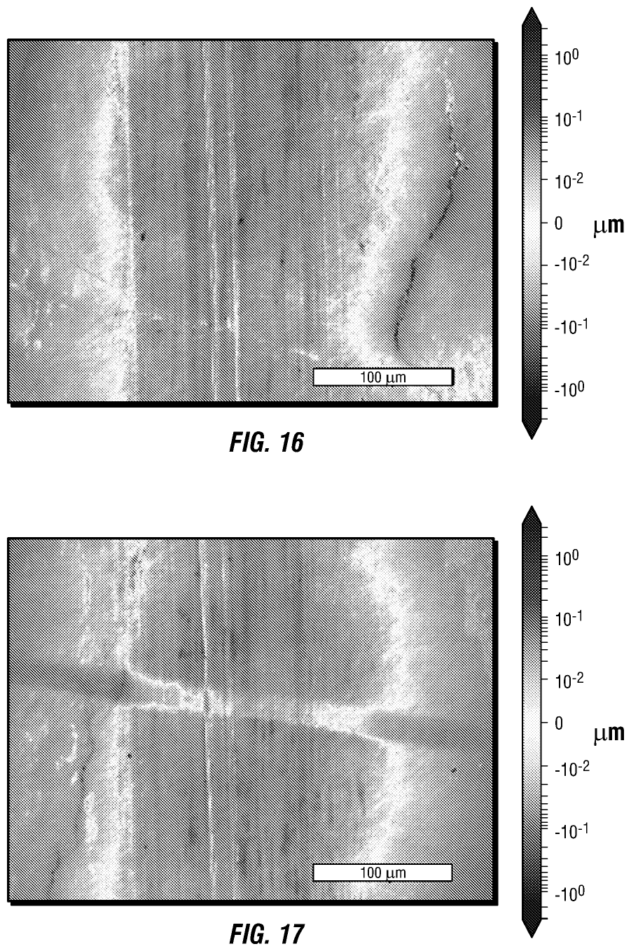

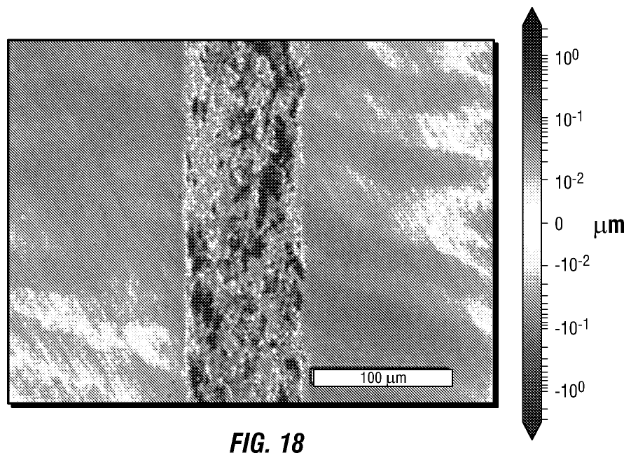

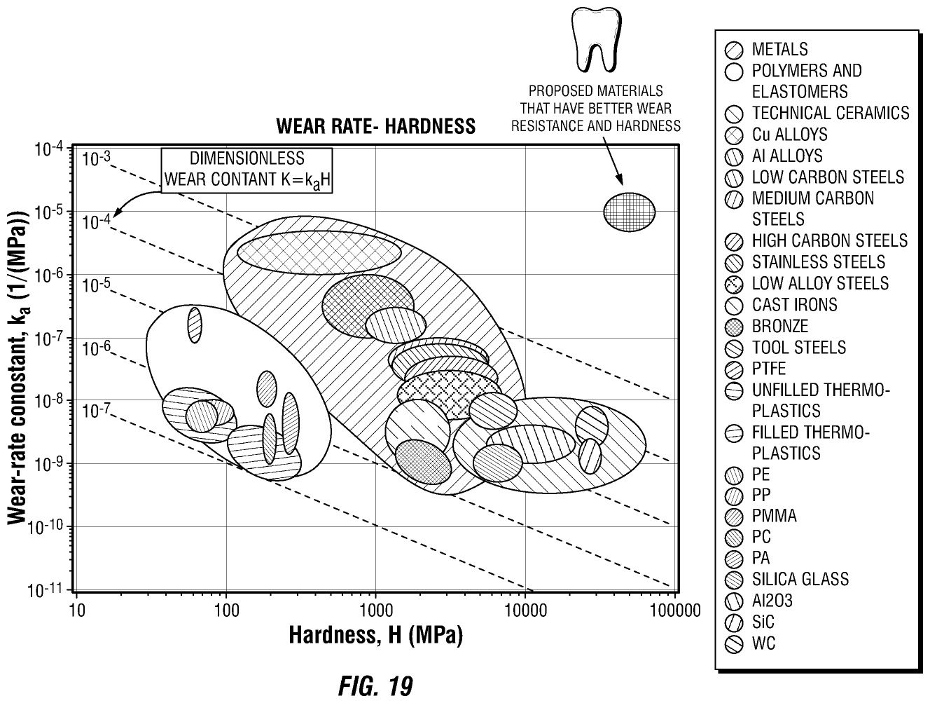

FIGS. 12A-B illustrate repairing film generation on dog teeth. The red color indicates high surface area. FIG. 13 illustrates a profile of dog teeth. FIGS. 14A-B illustrate that scratching does not remove the film. FIGS. 15A-B illustrate film generation on human teeth. FIG. 16 illustrates an area being coated by a film. FIG. 17 illustrates that scratching does not remove the film. FIG. 18 illustrates an image of a film built on a tooth. FIG. 19 illustrates proposed materials that have better wear resistance and hardness.

### Example 2.1. Greenies Dental Treats Original Flavor for Dogs

Ingredients: Wheat Flour, Glycerin, Wheat Gluten, Gelatin, Water, Powdered Cellulose, Lecithin, Minerals (Dicalcium Phosphate, Potassium Chloride, Calcium Carbonate, Magnesium Amino Acid Chelate, Zinc Amino Acid Chelate, Iron Amino Acid Chelate, Copper Amino Acid Chelate, Manganese Amino Acid Chelate, Selenium, Potassium Iodide), Natural Poultry Flavor, Choline Chloride, Fruit Juice Color, Vitamins (Dl-Alpha Tocopherol Acetate [Source Of Vitamin E], Vitamin B12 Supplement, D-Calcium Pantothenate [Vitamin B5], Niacin Supplement, Vitamin A Supplement, Riboflavin Supplement [Vitamin B2], Vitamin D3 Supplement, Biotin, Thiamine Mononitrate [Vitamin B1], Pyridoxine Hydrochloride [Vitamin B6], Folic Acid), and Turmeric Color.

### Example 2.2. Greenies Dental Treats Blueberry Flavor for Dogs

Ingredients: Wheat flour, glycerin, wheat gluten, gelatin, water, powdered cellulose, lecithin, natural flavors, minerals (dicalcium phosphate, potassium chloride, calcium carbonate, magnesium amino acid chelate, zinc amino acid chelate, iron amino acid chelate, copper amino acid chelate, manganese amino acid chelate, selenium, potassium iodide), dried blueberries, choline chloride, fruit juice color, vitamins (dl-alpha tocopherol acetate [source of vitamin E], vitamin B12 supplement, d-calcium pantothenate [vitamin B5], niacin supplement, vitamin A supplement, riboflavin supplement [vitamin B2], vitamin D3 supplement, biotin, thiamine mononitrate [vitamin B1], pyridoxine hydrochloride [vitamin B6], folic acid), and turmeric color.

### Example 2.3. Greenies Dental Treats “Fresh” Flavor for Dogs

Ingredients: Wheat Flour, Glycerin, Wheat Gluten, Gelatin, Water, Powdered Cellulose, Lecithin, Natural Flavors, Minerals (Dicalcium Phosphate, Potassium Chloride, Calcium Carbonate, Magnesium Amino Acid Chelate, Zinc Amino Acid Chelate, Iron Amino Acid Chelate, Copper Amino Acid Chelate, Manganese Amino Acid Chelate, Selenium, Potassium Iodide), Dried Spearmint, Choline Chloride, Fruit Juice Color, Vitamins (Dl-Alpha Tocopherol Acetate [Source Of Vitamin E], Vitamin B12 Supplement, D-Calcium Pantothenate [Vitamin B5], Niacin Supplement, Vitamin A Supplement, Riboflavin Supplement [Vitamin B2], Vitamin D3 Supplement, Biotin, Thiamine Mononitrate [Vitamin B1], Pyridoxine Hydrochloride [Vitamin B6], Folic Acid), and Turmeric Color.

### Example 2.4. Greenies Dental Treat Tuna Flavor for Cats

Ingredients: Chicken Meal, Rice Flour, Wheat Flour, Corn Gluten Meal, Oat Fiber, Poultry Fat, (Preserved With Mixed Tocopherols), Natural Poultry Flavor, Sodium Gluconate, Tuna Flavor, Ground Flaxseed, Brewer's Dried Yeast, Ground Flaxseed, Calcium Carbonate, Sodium Chloride, Potassium Chloride, Zinc Sulfate, Ferrous Sulfate, Copper Sulfate, Manganese Oxide, Calcium Iodate, Sodium Selenite, Cobalt Carbonate, Vitamin A Supplement, Vitamin D3 Supplement, Vitamin E Supplement, Niacin, D-calcium Pantothenate, ThiamineMononitrate, Riboflavin Supplement, Pyridoxine Hydrochloride, Folic Acid, Menadione Sodium Bisulfite Complex, Biotin, Vitamin B12 Supplement,) Citric Acid, Taurine, Mixed Tocopherols and Sodium Copper Chlorophyllin.

### Example 2.5. Greenies Dental Treat Salmon Flavor for Cats

Ingredients: Chicken Meal, Rice Flour, Wheat Flour, Corn Gluten Meal, Oat Fiber, Poultry Fat, (Preserved With Mixed Tocopherols), Salmon Meal, Natural Poultry Flavor, Sodium Gluconate, Ground Flaxseed, Brewer's Dried Yeast, Ground Flaxseed, Calcium Carbonate, Sodium Chloride, Potassium Chloride, Zinc Sulfate, Ferrous Sulfate, Copper Sulfate, Manganese Oxide, Calcium Iodate, Sodium Selenite, Cobalt Carbonate, Vitamin A Supplement, Vitamin D3 Supplement, Vitamin E Supplement, Niacin, D-calcium Pantothenate, Thiamine Mononitrate, Riboflavin Supplement, Pyridoxine Hydrochloride, Folic Acid, Menadione Sodium Bisulfite Complex, Biotin, Vitamin B12 Supplement, Citric Acid, Taurine, Mixed Tocopherols, and Sodium Copper Chlorophyllin.

### Example 2.6. Pedigree Dental Stix Bacon Flavor for Dogs

Ingredients: Rice Flour, Wheat Starch, Glycerin, Gelatin, Gum Arabic, Calcium Carbonate, Natural Poultry Flavor, Powdered Cellulose, Sodium Tripolyphosphate, Iodized Salt, Potassium Chloride, Choline Chloride, L-Ascorbyl-2-Polyphosphate (Source of Vitamin C), Vitamin A Supplement, Niacin, D-Calcium Pantothenate, Folic Acid, Vitamin D3 Supplement, Vitamin B12 Supplement, Riboflavin (Vitamin B2), Pyridoxine Hydrochloride (Vitamin B6), DL-Alpha Tocopherol Acetate (Source of Vitamin E), Thiamine Mononitrate (Vitamin B1), Potassium Sorbate (Preservative), Smoke Flavor, Zinc Sulfate, Green Tea Extract, Turmeric, Iron Oxide and Copper Sulfate.

### Example 2.7. Blue Buffalo Dental Bones Natural Flavor

Ingredients: Potatoes, Powdered Cellulose, Vegetable Glycerin, Water, Gelatin, Pea Protein, Sunflower Lecithin, Natural Flavor, Oat Hulls, Sunflower Oil, Flaxseed, Carrot, Calcium Carbonate, Dehydrated Beets (added for color), Zinc Propionate, Blueberries, Parsley, Dehydrated Alfalfa Meal, preserved with Citric Acid and Mixed Tocopherols, and Oil of Rosemary.

### Example 3. Methods and Compositions for Tooth Whitening

In this Example, Applicants provide an object (e.g., a chewable object), including a composition associated with the object, where the composition is released onto a tooth (e.g., as the objected is chewed). Thereafter, the composition makes teeth white. The composition (i.e., the whitening material) can be added to objects (e.g., chewing objects, such as gum and candies, including gummy bears for example, or pastes or liquids). In this Example, the composition (i.e., whitening material) is aqueous paste containing a mixture of Zirconium oxide nanoparticles and M-600 intercalated ZrP nanoparticles. The composition (i.e., whitening material) can become effective once the application (e.g., chewing, rubbing, massaging and/or brushing) starts. The compositions (i.e., whitening materials) can be applied to teeth daily.

This Example also provides a method of whitening tooth by applying an object of the present disclosure to a tooth, where the object includes a composition of the present disclosure, where the applying releases the composition onto the tooth, and where the composition has whitening effects on the tooth. The applying includes chewing a chewable object that include a composition of the present disclosure, where the composition is released onto the tooth as the chewable object is chewed.

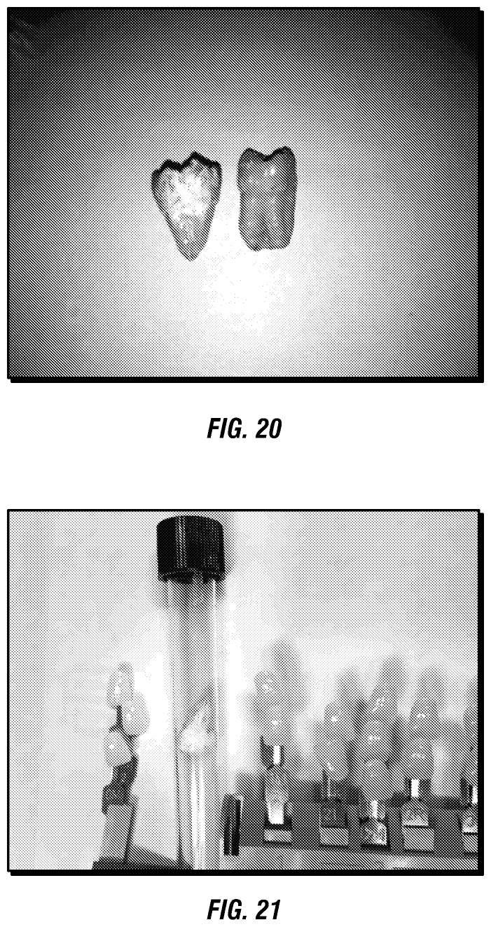

As shown in FIGS. 20-21, human teeth were repaired by rubbing the compositions of the present disclosure onto the tooth for a few minutes. The tooth appeared white compared to a regular extracted tooth.

Additional experimental results and examples are disclosed in U.S. Provisional Patent Application Nos. 62/806,488 and 62/966,691. The entirety of the experimental results in the aforementioned provisional patent applications are incorporated herein by reference.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of altering a tooth, said method comprising:
applying an object to the tooth, wherein the object comprises a composition,
wherein the composition is released from the object onto the tooth, and
wherein the composition has an altering effect on the tooth,
wherein the altering effect is selected from the group consisting of tooth whitening, tooth repair, tooth maintenance, or combinations thereof.

2. The method of claim 1,
wherein the object is selected from the group consisting of porous materials, woods, fabrics, chewable objects, chew toys, chew bones, treats, gums, objects capable of being applied topically by chewing rubbing or brushing, pastes, liquids, gels, powders, rinses, or combinations thereof; and
wherein the composition is selected from the group consisting of compositions with tooth whitening effects, compositions with tooth repair effects, compositions with tooth maintenance effects, hydrogen peroxide, carbamide peroxide, fluoride containing materials, phosphorous containing materials, calcium phosphate, zirconium phosphate (ZrP), α-zirconium phosphate, γ-zirconium phosphate, titanium phosphate, γ-titanium phosphate, iron oxide, zirconium oxide, zirconium dioxide, hydroxyapatite (HAP), kaolinite, bentonite, gold, silver, silica, ceria, alumina, zirconia, calcium aluminate, boron carbide, silicon carbide, silicon nitride, iron oxide, magnesium oxides, zinc chloride, sodium fluoride, hydrated salts thereof, amine-intercalated materials thereof, compositions comprising an organic:inorganic mass ratio of about 0.1:1 to about 0.1:100, compositions comprising an organic:inorganic mass ratio of about 0.1:1 to about 1:2, or combinations thereof.

3-10. (canceled)

11. The method of claim 1, wherein the composition comprises an amine-intercalated zirconium phosphate.

12. The method of claim 11, wherein the amine comprises an amine-based polymer.

13. The method of claim 11, wherein the amine-based polymer is selected from the group consisting of polyetheramines, Jeffamines, Jeffamine M600, or combinations thereof.

14. The method of claim 1, wherein the composition is in the form of particles, wherein the particles are selected from the group consisting of calcium phosphate particles, zirconium dioxide particles, hydroxyapatite particles, kaolinite particles, bentonite particles, cloisite particles, gold particles, silver particles, silica particles, ceria particles, alumina particles, zirconia particles, calcium aluminate particles, boron carbide particles, silicon carbide particles, silicon nitride particles, iron oxide particles, magnesium oxide particles, zinc chloride particles, sodium fluoride particles, zirconium phosphates particles, zirconium phosphate particles, titanium phosphate particles, hydrated salts thereof, or combinations thereof.

15. (canceled)

16. The method of claim 14, wherein the particles comprise amine-intercalated zirconium phosphate particles.

17. The method of claim 14, wherein the particles are functionalized with a functionalizing agent, wherein the functionalizing agent is an organic molecule selected from the group consisting of polymers, amine-based polymers, polyethylene glycols, surfactants, erythritol, sorbitol, glycerol, flavorants, triclosan, sodium lauryl sulfate, or combinations thereof.

18-20. (canceled)

21. The method of claim 1, wherein the object comprises a second composition, wherein the second composition is capable of enticing chewing of the object.

22-23. (canceled)

24. The method of claim 1, wherein the applying comprises chewing the object to thereby release the object onto the tooth as the object is chewed, rubbing the object onto the tooth to thereby release the object onto the tooth as the object is rubbed onto the tooth, brushing the object onto the tooth to thereby release the object onto the tooth as the object is brushed onto the tooth, rinsing the tooth with the object, or combinations thereof.

25-26. (canceled)

27. The method of claim 1, wherein the composition forms a solid film on a surface of the tooth.

28. The method of claim 27, wherein the film has a thickness ranging from 100 nm to 5 μm.

29. The method of claim 27, wherein the film is in the form of a tribofilm on a surface of the tooth.

30. The method of claim 1, wherein the altering effect comprises at least one of:
tooth maintenance, and wherein the tooth maintenance comprises protection from cracks, improvement of surface integrity, prevention of tooth breakdown, providing antimicrobial properties, or combinations thereof;
tooth repair, and wherein the tooth repair comprises the filling of cracks, tooth restoration, or combinations thereof; or
tooth whitening.

31-32. (canceled)

33. The method of claim 1, wherein the object is applied to the tooth of a subject, wherein the subject is selected from the group consisting of a dog, a cat, a rat, a gerbil, a hamster, a guinea pig, a rabbit, a human, or combinations thereof.

34. (canceled)

35. An object comprising a composition,
wherein the composition is releasable from the object onto a tooth, and
wherein the composition has altering effects on the tooth,
wherein the altering effects are selected from the group consisting of tooth whitening, tooth repair, tooth maintenance, or combinations thereof.

36. The object of claim 35,
wherein the object is selected from the group consisting of porous materials, woods, fabrics, chewable objects, chew toys, chew bones, treats, gums, gels, powders, objects capable of being applied topically by chewing rubbing or brushing, pastes, liquids, rinses, or combinations thereof; and
wherein the composition is selected from the group consisting of compositions with tooth whitening effects, compositions with tooth repair effects, compositions with tooth maintenance effects, hydrogen peroxide, carbamide peroxide, fluoride containing materials, phosphorous containing materials, calcium phosphate, zirconium phosphate (ZrP), α-zirconium phosphate, γ-zirconium phosphate, titanium phosphate, γ-titanium phosphate, iron oxide, zirconium oxide, zirconium dioxide, hydroxyapatite (HAP), kaolinite, bentonite, gold, silver, silica, ceria, alumina, zirconia, calcium aluminate, boron carbide, silicon carbide, silicon nitride, iron oxide, magnesium oxides, zinc chloride, sodium fluoride, hydrated salts thereof, amine-intercalated materials thereof, compositions comprising an organic:inorganic mass ratio of about 0.1:1 to about 0.1:100, compositions comprising an organic:inorganic mass ratio of about 0.1:1 to about 1:2, or combinations thereof.

37-44. (canceled)

45. The object of claim 35, wherein the composition comprises an amine-intercalated zirconium phosphate.

46. The object of claim 45, wherein the amine comprises an amine-based polymer.

47. The object of claim 45, wherein the amine-based polymer is selected from the group consisting of polyetheramines, Jeffamines, Jeffamine M600, or combinations thereof.

48. The object of claim 35, wherein the composition is in the form of particles, wherein the particles are selected from the group consisting of calcium phosphate particles, zirconium dioxide particles, hydroxyapatite particles, kaolinite particles, bentonite particles, cloisite particles, gold particles, silver particles, silica particles, ceria particles, alumina particles, zirconia particles, calcium aluminate particles, boron carbide particles, silicon carbide particles, silicon nitride particles, iron oxide particles, magnesium oxide particles, zinc chloride particles, sodium fluoride particles, zirconium phosphates particles, zirconium phosphate particles, titanium phosphate particles, hydrated salts thereof, or combinations thereof.

49. (canceled)

50. The object of claim 48, wherein the particles comprise amine-intercalated zirconium phosphate particles.

51. The object of claim 48, wherein the particles are functionalized with a functionalizing agent, wherein the functionalizing agent is an organic molecule selected from the group consisting of polymers, amine-based polymers, polyethylene glycols, surfactants, erythritol, sorbitol, glycerol, flavorants, triclosan, sodium lauryl sulfate, or combinations thereof.

52-53. (canceled)

54. The object of claim 35, wherein the object comprises a second composition, wherein the second composition is capable of enticing chewing of the object.

55-56. (canceled)
